(19) United States
(12) Patent Application Publication
(10) Pub. No.: US 2025/0241899 A1
(43) Pub. Date: Jul. 31, 2025

(54) Topical Antifungal Non-Aqueous Formulations and Preparation Methods Thereof

(71) Applicant: Hefei Cosource Pharmaceuticals Co., Ltd, Hefei (CN)

(72) Inventors: Wei Wu, Hefei (CN); Dengjun Chen, Hefei (CN); Yuting Jin, Hefei (CN); Lingzhi Zhang, Hefei (CN); Jingjing Liu, Hefei (CN); Fulin Zhao, Hefei (CN); Xujun Fang, Hefei (CN); Xinghua Dong, Hefei (CN)

(73) Assignee: Hefei Cosource Pharmaceuticals Co., Ltd, Hefei (CN)

(21) Appl. No.: 19/037,494

(22) Filed: Jan. 27, 2025

(30) Foreign Application Priority Data
Jan. 29, 2024 (CN) 202410124988.6

(51) Int. Cl. A61K 31/4196; A61K 9/00; A61K 31/427; A61K 31/506; A61K 47/02

(52) U.S. Cl. CPC A61K 31/4196; A61K 9/0019; A61K 9/0046; A61K 9/0048; A61K 31/427; A61K 31/506; A61K 47/02

(57) ABSTRACT

Provided are a pharmaceutical composition for topical administration for the treatment of eye diseases and ear diseases caused by fungal infections, such as fungal keratitis, otitis externa or otitis media caused by fungal infections, and a preparation method thereof. The pharmaceutical composition comprises a triazole antifungal agent as an active ingredient, a non-aqueous solvent, and optionally a tonicity regulator, and may be a non-aqueous preservative-free composition. The non-aqueous composition of the present disclosure has one or more of the following advantages: high stability, preservative-free, low irritation, increased concentration of the antifungal agent in local tissues, reduced systemic toxicity and side effects, increased retention time of the antifungal agent in local tissues, reduced administration frequency, and improved patient compliance.

### CROSS-REFERENCE TO RELATED APPLICATIONS

The present application claims the benefit and priority to the Chinese patent application No. 202410124988.6 filed before the China national intellectual property administration on Jan. 29, 2024, the content of which is incorporated herein by reference in its entirety.

### TECHNICAL FIELD

The present disclosure belongs to the field of pharmaceutical formulations, and specifically relates to a topical pharmaceutical formulation for the treatment of eye diseases and ear diseases caused by fungal infections, and a preparation method thereof.

### BACKGROUND

Fungal keratitis (FK) is a serious blinding keratopathy caused by direct fungal infection of the cornea, and is the leading cause of blindness in infectious keratopathy. Fungal keratitis requires long-term administration of a drug to the ocular surface. Two antifungal agents, amphotericin B and natamycin, are used in the treatment of fungal keratitis. The currently used 0.25% amphotericin B eye drop needs to be formulated with an injection liquid for immediate use due to its unstablity and sensitivity to heat and light, greatly limiting its clinical application. The clinically used 5% natamycin suspension eye drop has a high manufacture cost and expensive price, and needs to be administered every 1 to 2 hours.

In addition, an aqueous ophthalmic formulation will activate the blink reflex after drip administration to an eye, with most of the topically administrated drug being washed away within 15 to 30 seconds. Meanwhile, the high surface tension of water-based droplets further hinders the diffusion of water-based droplets on the ocular surface. Therefore, the traditional water-based ophthalmic formulations have low bioavailability, even as low as 3% to 4%.

In clinical practice, a fluconazole injection is used topically through ear canal irrigation to treat fungal otitis externa or otitis media. Currently, there is no specifically designed otic formulations for the treatment of fungal otitis externa or otitis media.

Voriconazole is a broad-spectrum antifungal agent of the triazole class, and it is currently available in injections, oral suspensions, and oral tablets. However, there is no commercially available ophthalmic or otic formulations of voriconazole.

Voriconazole has an extremely slight solubility in water, with a solubility of 0.61 mg/ml at pH 7 and 0.2 mg/ml at pH 3. Voriconazole undergoes retrograde aldol condensation reaction in water, resulting in inactive enantiomers. The hydroxyl group on voriconazole is protonated in proton solvents (such as water, alcohols, etc.), accelerating degradation, and can also be destabilized by increased hydrogen-oxygen dipolarization of the hydroxyl group in polar, non-proton solvents. Voriconazole is unstable under both alkaline and strong acid conditions, with a log D of 1.8, and its semi-polarity nature causes some difficulties in selecting a suitable solubilization means. It has been reported that unencapsulated voriconazole is immobilized in an inclusion complex by the addition of a crosslinking agent in association with a hydrogen bond on hydroxypropyl beta-cyclodextrin using a cyclodextrin inclusion technique. However, the preparation steps of this method are complicated, and the effect of improving the stability of voriconazole solution is not significant.

Therefore, there is an urgent need for a long-acting, stable and topical formulation of an antifungal agent in clinical practice.

### SUMMARY OF INVENTION

In one aspect, the present disclosure provides a pharmaceutical composition, comprising: a triazole antifungal agent as an active ingredient, a non-aqueous solvent, and optionally a tonicity regulator.

In another aspect, the present disclosure provides a method for preparing a pharmaceutical composition comprising a triazole antifungal agent as an active ingredient, a non-aqueous solvent, and optionally a tonicity regulator, comprising mixing the triazole antifungal agent with the non-aqueous solvent and then optionally adding the tonicity regulator.

In still another aspect, the present disclosure provides a use of a pharmaceutical composition comprising a triazole antifungal agent as an active ingredient, a non-aqueous solvent, and optionally a tonicity regulator in the manufacture of a medicament for the treatment of eye diseases and ear diseases caused by fungal infections, such as fungal keratitis, otitis externa or otitis media caused by fungal infections.

### BRIEF DESCRIPTION OF DRAWINGS

FIG. 1 shows the changes in content for a voriconazole eye drop and voriconazole injection prepared according to Example 1 and Comparative Example 1 during the 0 to 2 months of the acceleration experiment.

FIG. 2 shows the changes in total impurities of related substances for a voriconazole eye drop and voriconazole injection prepared according to Example 1 and Comparative Example 1 during the 0 to 2 months of the acceleration experiment.

FIG. 3 shows the changes in maximum single impurity of related substances for a voriconazole eye drop and voriconazole injection prepared according to Example 1 and Comparative Example 1 during the 0 to 2 months of the acceleration experiment.

FIG. 4 shows a logarithmic plot of plasma concentration-time curves in the plasma of rabbits for a voriconazole eye drop and voriconazole injection prepared according to Example 1 and Comparative Example 1.

FIG. 5 shows a logarithmic plot of drug concentration-time curves in the aqueous humor of rabbits for a voriconazole eye drop and voriconazole injection prepared according to Example 1 and Comparative Example 1.

FIG. 6 shows a logarithmic plot of drug concentration-time curves in the cornea of rabbits for a voriconazole eye drop and voriconazole injection prepared according to Example 1 and Comparative Example 1.

FIG. 7 shows a logarithmic plot of drug concentration-time curves in the conjunctiva of rabbits for a voriconazole eye drop and voriconazole injection prepared according to Example 1 and Comparative Example 1.

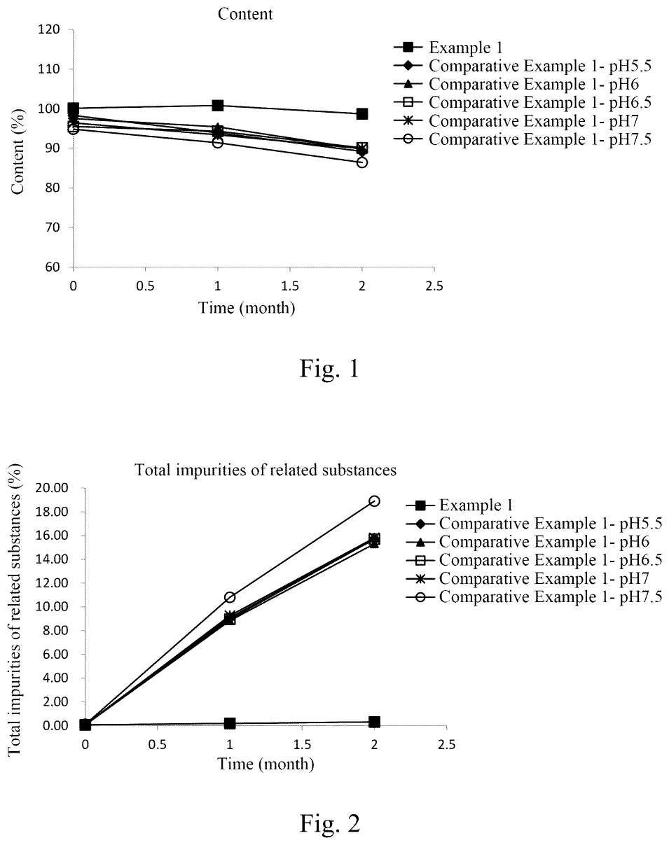

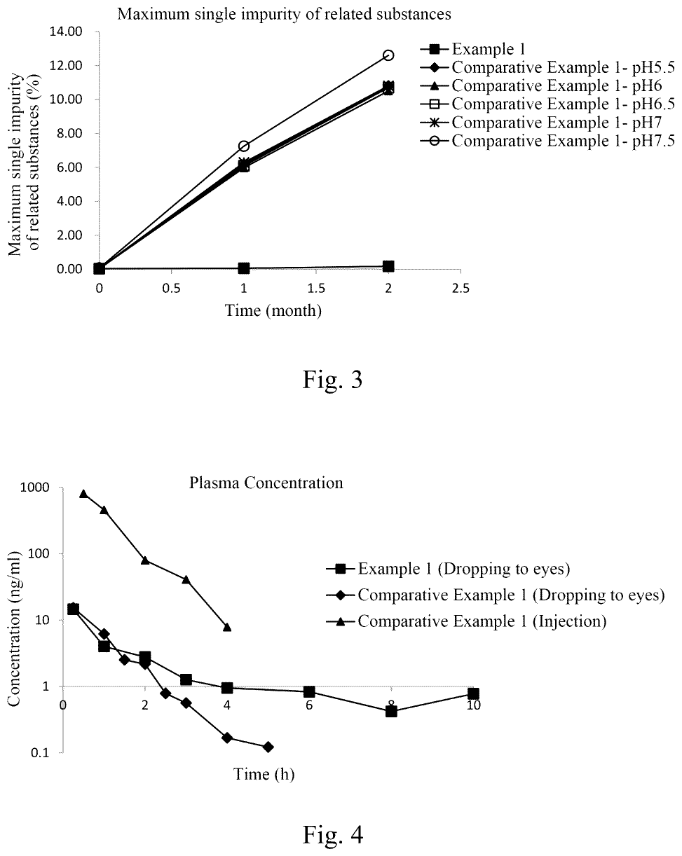

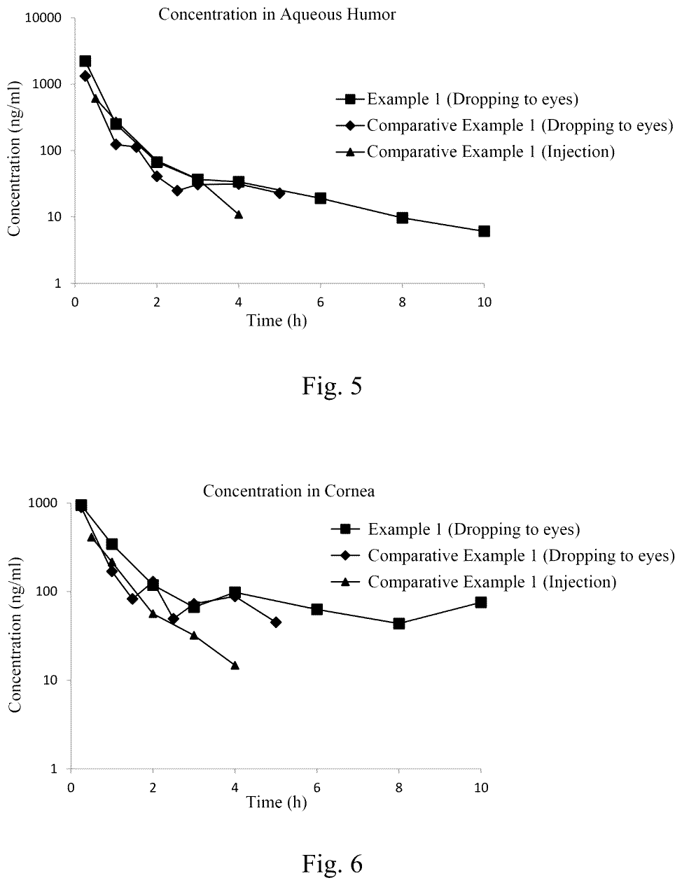

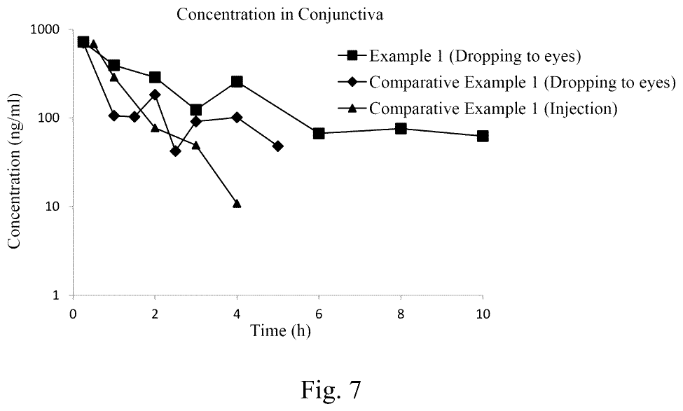

### DESCRIPTION OF EMBODIMENTS

In one aspect, the present disclosure provides a pharmaceutical composition comprising a triazole antifungal agent as an active ingredient, a non-aqueous solvent, and optionally a tonicity regulator.

In some embodiments, the pharmaceutical composition is non-aqueous. In some embodiments, the pharmaceutical composition does not contain a preservative. In some embodiments, the pharmaceutical composition is a non-aqueous preservative-free composition.

Preservative is often regarded as an unwanted necessity, preserving the formulation but with deleterious effects on the eyes. One common solution to the problem of preservation is to dispense the product in a unit dose form. However, unit dose packaging is significantly more expensive than multi-dose packaging, adding to the cost of the final product. The pharmaceutical composition of the present disclosure is a non-aqueous formulation, and the need for an anti-infective preservative is removed. Therefore, the pharmaceutical composition of the present disclosure is allowed to use multiple dose delivery from a single applicator, which is easier for carrying and travel around. The non-aqueous preservative-free pharmaceutical composition has been shown not to cause corneal damage. Furthermore, the non-aqueous formulation can avoid hydrolysis and oxidation reactions, thereby improving the stability of the formulation and avoiding possible tip blockage.

In some embodiments, the triazole antifungal agent is a triazole-based antifungal agent, examples of which include but are not limited to fluconazole, voriconazole, and isavuconazole. In some embodiments, the triazole antifungal agent is voriconazole.

In some embodiments, the non-aqueous solvent is a glyceride of a C₆-C₁₂ fatty acid, or the non-aqueous solvent contains a glyceride of a C₆-C₁₂ fatty acid. In some embodiments, the glyceride of the C₆-C₁₂ fatty acid is a glyceride of a saturated C₆-C₁₂ fatty acid and/or a glyceride of an unsaturated C₆-C₁₂ fatty acid. In some embodiments, the glyceride of the fatty acid is selected from the group consisting of monoglycerides, diglycerides, triglycerides of saturated or unsaturated C₆-C₁₂ fatty acids, and any combination thereof.

In some embodiments, the non-aqueous solvent has an ester bond (—C(O)—O—) content of about 20.0% to about 35.0% by molecular weight, or about 22.0% to about 28.0% by molecular weight. In some embodiments, the glyceride of the C₆-C₁₂ fatty acid has an ester bond (—C(O)—O—) content of about 20.0% to about 35.0% by molecular weight, or about 22.0% to about 28.0% by molecular weight. For example, the molecular weight of glycerol trihexanoate is 386.5, of which the molecular weight of the three ester bonds (—C(O)—O—) is 132, and accordingly, glycerol trihexanoate has the ester bond content of 34.2% by molecular weight.

In some embodiments, a fatty acid moiety in the glyceride of the C₆-C₁₂ fatty acid is one or more selected from the group consisting of hexanoic acid, heptanoic acid, octanoic acid, nonanoic acid, decanoic acid, undecanoic acid, and dodecanoic acid. In some embodiments, the glyceride of the C₆-C₁₂ fatty acid is selected from the group consisting of monoglycerides, diglycerides, and triglycerides of hexanoic acid, heptanoic acid, octanoic acid, nonanoic acid, decanoic acid, undecanoic acid, and dodecanoic acid, and any combination thereof. In some embodiments, the glyceride of the C₆-C₁₂ fatty acid is selected from the group consisting of triglycerides of hexanoic acid, heptanoic acid, octanoic acid, nonanoic acid, decanoic acid, undecanoic acid, and dodecanoic acid, and any combination thereof.

In some embodiments, the non-aqueous solvent is one or more selected from the group consisting of medium chain triglycerides, glycerol trihexanoate, glycerol trioctanoate, glycerol tridecanoate, and glycerol tridodecanoate.

In some embodiments, the medium chain triglyceride is one or more selected from the group consisting of triglycerides of octanoic acid, nonanoic acid, decanoic acid, undecanoic acid, and dodecanoic acid. In some embodiments, the medium chain triglyceride is glycerol trioctanoate, glycerol tridecanoate or a glyceride of a mixture of saturated octanoic acid and decanoic acid.

In some embodiments, the pharmaceutical composition further comprises the tonicity regulator. In some embodiments, the tonicity regulator is an agent for reducing tonicity.

In some embodiments, the tonicity regulator is cyclomethicone. In some embodiments, the cyclomethicone is a mixture of cyclicdimethylpolysiloxane compounds. It is a fully methylated cyclosiloxane containing a repeating unit represented by a molecular formula of [—(CH₃)₂SiO-]n, where n is 4, 5, or 6, or a mixture of the foregoing. It is a colorless, odorless, transparent, and non-greasy organosilicone liquid with lower viscosity and surface tension, as well as relatively high vapor pressure. The changes in the volatility of cyclomethicone can be achieved through mixing different polymers thereof, which exhibit subtle variations in properties, such as molecular weight, solubility, specific gravity, viscosity, and so on.

The physicochemical properties of four cyclomethicones from Dow Corning Company are listed exemplarily in the following table.

| | 244 | 245 | 344 | 345 |
|---|---|---|---|---|
| Properties | Liquid | Liquid | Liquid | Liquid |
| Ingredient (polymer %) | 90% n = 4 | 95% n = 5 | 90% n = 4 | 75% n = 5 |
| | | | 10% n = 5 | 25% n = 6 |
| Refractivity (25° C.) | 1.394 | 1.397 | 1.394 | 1.398 |
| Viscosity (cs, 25° C.) | 2.5 | 4.2 | 2.5 | 5 |
| Specific gravity (25° C.) | 0.953 | 0.956 | 0.85 | 0.956 |
| Surface tension (25° C., | 17.8 | 18 | 19 | 20.8 |
| dyn/cm) | | | | |
| Flash point (° C.) (closed | 55 | 76 | 52 | 74 |
| loop) | | | | |
| Boiling point (° C., 760 | 172 | 205 | 178 | 217 |
| mm Hg) | | | | |

Studies have shown that undiluted cyclomethicone produces little or no irritation when applied to both intact and broken skins of rabbits. Eye studies have shown that cyclomethicone only produces a mild and transient conjunctival irritation in both irrigated and unirrigated eyes, with symptoms completely subsiding within 24 hours and no corneal damage. Cyclomethicone is nonirritating or nonallergenic to human skins. Cyclomethicone is very safe for application based on available data.

In some embodiments, the pharmaceutical composition comprises about 0.2% (w/v) to about 2% (w/v) of the triazole antifungal agent, about 80% (v/v) to about 100% (v/v) of the non-aqueous solvent, and about 0% (v/v) to about 20% (v/v) of the tonicity regulator.

In some embodiments, the content of the triazole antifungal agent in the pharmaceutical composition is about 0.2% (w/v) to about 2% (w/v), about 0.5% (w/v) to about 2% (w/v), about 0.5% (w/v) to about 1.5% (w/v), about 0.5% (w/v) to about 1.0% (w/v), or about 1.0% (w/v) to about 1.5% (w/v).

In some embodiments, the content of the non-aqueous solvent in the pharmaceutical composition is about 80% (v/v) to about 100% (v/v), about 80% (v/v) to about 99% (v/v), about 80% (v/v) to about 98% (v/v), about 90% (v/v) to about 100% (v/v), about 90% (v/v) to about 99% (v/v), or about 90% (v/v) to about 98% (v/v).

In some embodiments, the content of the tonicity regulator in the pharmaceutical composition is about 0% (v/v) to about 20% (v/v), about 0% (v/v) to about 10% (v/v), about 2% (v/v) to about 20% (v/v), or about 2% (v/v) to about 10% (v/v).

In some embodiments, the pharmaceutical composition is in a liquid form, such as eye drops or ear drops.

In another aspect, the present disclosure provides a method for preparing the pharmaceutical composition comprising the triazole antifungal agent as the active ingredient, the non-aqueous solvent, and optionally the tonicity regulator, comprising mixing the triazole antifungal agent with the non-aqueous solvent and optionally adding the tonicity regulator.

In another aspect, the present disclosure provides the use of the pharmaceutical composition comprising the triazole antifungal agent as the active ingredient, the non-aqueous solvent, and optionally the tonicity regulator in the manufacture of a medicament for the treatment of eye diseases and ear diseases caused by fungal infections, such as fungal keratitis, otitis externa or otitis media caused by fungal infections.

The inventors of the present disclosure have found that the extraordinary dispersion property of oily matrices, and especially specific fatty acid glycerides, support the distribution of triazole antifungal agents on the corneal surface, and that the oily matrices produce much smaller droplet sizes due to their low viscosity and low surface tension compared with aqueous matrices, which avoids liquid overflow and immediate loss of most of the dosages, thereby improving the bioavailability of the drugs.

The non-aqueous pharmaceutical formulation of the present disclosure has one or more of the following advantages: high stability, preservative-free, low irritation, increased concentration of the antifungal agent in local tissues, reduced systemic toxicity and side effects, as well as increased retention time of the antifungal agent in local tissues, reduced administration frequency, and improved patient compliance.

### EXAMPLES

The present disclosure will be described in further details below in conjunction with specific examples. These examples may be modified to obtain other embodiments without departing from the scope or spirit of the present disclosure. Therefore, the following examples are non-limitative.

Unless otherwise specified, all numbers used in the Specification and Claims to represent feature sizes, quantities, and physicochemical properties should be understood as being modified by the term “about” in all cases. Therefore, unless otherwise stated to the contrary, the numerical parameters listed in the foregoing Specification and the appended claims are approximations, and those skilled in the art can utilize the teachings disclosed herein to seek the desired characteristics, and appropriately change these approximations. The use of a numerical range represented by endpoints includes all numbers within said range and any range within said range, for example, 1 to 5 includes 1, 1.1, 1.3, 1.5, 2, 2.75, 3, 3.80, 4, and 5, etc.

The drugs or reagents used in the present disclosure are all conventional commercially-available products, unless otherwise specified.

### Comparative Example 1 (Patent Application Publication No. CN110812323 A)

| Components | Amount | |
|---|---|---|
| Voriconazole | 1 | g |
| Hydroxypropyl β-cyclodextrin (HP-β-CD) | 20 | g |
| Polyvinyl alcohol PVA | 0.6 | g |
| Glycine | 0.1 | g |
| Hydrochloric acid | appropriate amount | |
| Sodium chloride | 0.1 | g |
| Total Volume | 100 | ml |

### Preparation Process:

1. HP-β-CD was added to 40 ml of water and dissolved to obtain a clear solution, the pH of the solution was adjusted to pH 5.0 with HCl, and then the prescribed amount of NaCl was added and dissolved;
2. Voriconazole was added to the above solution, stirred for 3 hours, and then dissolved to obtain a clear solution;
3. PVA was added, stirred for 30 minutes, and dissolved to obtain a clear solution, and then glycine was added, dissolved and stirred for 30 minutes to stabilize the solution;
4. The resulting solution was transferred to a 100 ml volumetric flask, made up the volume with water, and filtered with a 0.22 μm water-system filter head; and
5. The filtrate was divided into 5 portions, and the pH was adjusted to 5.5, 6, 6.5, 7, and 7.35 with HCl in order.

### Comparative Example 2 (Glycerol Triacetate)

| Components | Amount | |
|---|---|---|
| Voriconazole | 1 | g |
| Glycerol triacetate | 49 | ml |
| Total volume | 50 | ml |

### Preparation Process:

1. Voriconazole was added to glyceryl triacetate, sealed and filled with nitrogen gas, and then dissolved by heating at a temperature of 60° C. to 65° C.; and
2. The resulting oil solution of voriconazole was cooled to room temperature, and then filtered using a 0.22 μm nylon filter head to obtain a formulation.

The oil solution was dropped into the eyes of rabbits, and was found to cause strong irritation to the ocular surface as well as messy fur of the rabbits.

### Comparative Example 3 (Glycerol Tributyrate)

| Components | Amount | |
|---|---|---|
| Voriconazole | 1 | g |
| Glycerol tributyrate | 49 | ml |
| Cyclomethicone | 1 | ml |
| Total volume | 50 | ml |

### Preparation Process:

1. Voriconazole was added to glyceryl tributyrate, sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain a formulation.

The oil solution was dropped onto the surface of human skins, and was found to produce an irritating odor and was not suitable for use as an eye drop or an ear drop.

### Comparative Example 4 (Castor Oil)

| Components | Amount | |
|---|---|---|
| Voriconazole | 0.5 | g |
| Castor oil | 49 | ml |
| Cyclomethicone | 1 | ml |
| Total volume | 50 | ml |

### Preparation Process:

1. Voriconazole was added to castor oil, sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain a formulation.
Test results: After 24 hours of standing, the solution was observed to be in an unclarified state with precipitation of solutes.

### Comparative Example 5 (Soybean Oil)

| Components | Amount | |
|---|---|---|
| Voriconazole | 0.5 | g |
| Soybean oil | 49 | ml |
| Cyclomethicone | 1 | ml |
| Total volume | 50 | ml |

### Preparation Process:

1. Voriconazole was added to soybean oil, sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain a formulation.
Test results: After 24 hours of standing, the solution was observed to be in an unclarified state with precipitation of solutes.

### Comparative Example 6 (Sesame Oil)

| Components | Amount | |
|---|---|---|
| Voriconazole | 0.5 | g |
| Sesame oil | 49 | ml |
| Cyclomethicone | 1 | ml |
| Total volume | 50 | ml |

### Preparation Process:

1. Voriconazole was added to sesame oil, sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain a formulation.

Test results: After 24 hours of standing, the solution was observed to be in an unclarified state with precipitation of solutes.

### Example 1

| Components | Amount | |
|---|---|---|
| Voriconazole | 0.5 | g |
| Medium chain triglyceride MCT | 50 | ml |
| Total volume | 50 | ml |

### Preparation Process:

1. Voriconazole was added to a medium chain triglyceride solvent (Liaoning Shinsun Pharmaceutical Co., Ltd., batch number: 210502-2-01), sealed and filled with nitrogen gas, and then dissolved by heating at a temperature of 60° C. to 65° C.; and
2. The resulting oil solution of voriconazole was cooled to room temperature, and then filtered using a 0.22 μm nylon filter head to obtain Formulation 1.

### Example 2

| Components | Amount | |
|---|---|---|
| Voriconazole | 0.75 | g |
| Gglycerol trihexanoate | 49 | ml |
| Cyclomethicone | 1 | ml |
| Total volume | 50 | ml |

### Preparation Process:

1. Voriconazole was added to glycerol trihexanoate, sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain Formulation 2.

### Example 3

| Components | Amount | |
|---|---|---|
| Voriconazole | 0.5 | g |
| Glycerol tridecanoate | 49 | ml |
| Cyclomethicone | 1 | ml |
| Total volume | 50 | ml |

### Preparation Process:

1. Voriconazole was added to glycerol tridecanoate, sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain Formulation 3.

### Example 4

| Components | Amount | |
|---|---|---|
| Voriconazole | 1 | g |
| Glycerol trioctanoate | 45 | ml |
| Cyclomethicone | 5 | ml |
| Total volume | 50 | ml |

### Preparation Process:

1. Voriconazole was added to glycerol trioctanoate, sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain Formulation 4.

### Example 5

| Components | Amount | |
|---|---|---|
| Voriconazole | 0.25 | g |
| Medium chain triglyceride MCT | 40 | ml |
| Cyclomethicone | 10 | ml |
| Total volume | 50 | ml |

### Preparation Process:

1. Voriconazole was added to a medium chain triglyceride solvent (Liaoning Shinsun Pharmaceutical Co., Ltd., batch number: 210502-2-01), sealed and filled with nitrogen gas, and then dissolved by heating at a temperature of 60° C. to 65° C.; and
2. The resulting oil solution of voriconazole was cooled to room temperature, and cyclomethicone was added and stirred evenly, and then filtered using a 0.22 μm nylon filter head to obtain Formulation 5.

### Performance Tests

Unless otherwise specified, the amount of voriconazole was detected using the following high-performance liquid chromatography conditions:

Chromatographic conditions for content determination and related substance inspection: according to the high-performance liquid chromatography method (General Rule 0512) in Part IV of Chinese Pharmacopoeia, 2020 edition, octadecylsilane-bonded silica was used as the filler (4.6 mm×250 mm, 5 Mm or equivalent chromatographic column); 0.02 mol/L ammonium acetate buffer (adjusted to pH 4.0±0.3 with acetic acid)-methanol-acetonitrile (55:15:30) were used as as the mobile phase; the column temperature was 35° C., and the detection wavelength was 256 nm.

### Chromatographic Conditions for Pharmacokinetic Determination

Detection system: HPLC-UV (Shimadzu SPD-10AVP, LC-20AD)
Liquid chromatography column: Luna Omega C18 50×2.1 mm 1.6 μm S/N: H19-309764
Column temperature: 40° C.
Needle wash: isopropanol:methanol:water=1:1:1
Flow rate: 0.5 mL/min
Autosampler temperature: 4° C.
Injection volume: 10 μL
Mobile phase A: 0.1% aqueous solution of formic acid
Mobile phase B: methanol

### Gradient Elution:

| Time (min) | Units | Command | Value |
|---|---|---|---|
| 0.00 | Column oven | CTO.RVL | 1 |
| 0.40 | Column oven | CTO.RVL | 0 |
| 1.50 | Pump | B.Conc | 100 |
| 1.50 | Pump | B.Conc | 100 |
| 2.00 | Pump | B.Conc | 50 |
| 2.01 | Pump | Stop | — |
| 3.00 | Column oven | CTO.RVL | |

### Mass Spectrometry Conditions:

Mass spectrometry system: LC-MS/MS
Ionization method: (−) ESI
Scanning mode: MRM

### Parent Ions, Fragment Ions, and Collision Energy CE

| | Parent | Daughter | Retention | Q1 | | Q3 |
|---|---|---|---|---|---|---|
| | ions | ions | time | devi- | | devi- |
| Compounds | (m/z) | (m/z) | (ms) | ation | CE | ation |
| Voriconazole | 350.00 | 127.10 | 48.0 | −24.0 | −38.0 | −21.0 |
| | 350.00 | 281.10 | 48.0 | −17.0 | −17.0 | −28.0 |
| Verapamil | 455.35 | 303.15 | 100.0 | −21.0 | −30.0 | −22.0 |

### Test on Comparative Example 1 and Examples 1 to 5

The accelerated stability of the formulations in Comparative Example 1 and Examples 1 to 5 were determined.

Experimental method: The formulations prepared according to Comparative Example 1 and Examples 1 to 5 were placed under 40° C. and 75% RH conditions for 2 months. The contents of the active ingredient and related substances for each formulation were determined at 0, 1, and 2 months, respectively.

| | | | | | | | Maximum single | | | | | |
|---|---|---|---|---|---|---|---|---|---|---|---|---|
| | Voriconazole | | | Total impurities of | | | impurity of related | | | | | |
| | content % | | | related substances % | | | substances max % | | | Color and clarity degree of solution | | |
| Item | 0 | 1 | 2 | 0 | 1 | 2 | 0 | 1 | 2 | 0 | 1 | 2 |
| Sample | month | month | months | month | month | months | month | month | months | month | month | months |
| Example 1 | 100.1 | 100.8 | 98.7 | 0.06 | 0.18 | 0.31 | 0.04 | 0.0 | 0.17 | Clear | Clear | Clear |
| | | | | | | | | | | Transparent | Transparent | Transparent |
| Example 2 | 100.0 | 99.2 | 99.0 | 0.06 | 0.15 | 0.29 | 0.04 | 0.06 | 0.15 | Clear | Clear | Clear |
| | | | | | | | | | | Transparent | Transparent | Transparent |
| Example 3 | 99.5 | 99.6 | 99.0 | 0.07 | 0.18 | 0.30 | 0.04 | 0.07 | 0.17 | Clear | Clear | Clear |
| | | | | | | | | | | Transparent | Transparent | Transparent |
| Example 4 | 99.8 | 99.2 | 98.8 | 0.08 | 0.19 | 0.33 | 0.05 | 0.08 | 0.20 | Clear | Clear | Clear |
| | | | | | | | | | | Transparent | Transparent | Transparent |
| Example 5 | 99.8 | 99.9 | 99.0 | 0.07 | 0.20 | 0.35 | 0.05 | 0.08 | 0.19 | Clear | Clear | Clear |
| | | | | | | | | | | Transparent | Transparent | Transparent |
| Comparative | 98.3 | 93.9 | 89.2 | 0.06 | 9.08 | 15.70 | 0.04 | 6.19 | 10.80 | Clear | Clear | Clear |
| Example 1 | | | | | | | | | | Transparent | Transparent | Transparent |
| pH5.5 | | | | | | | | | | | | |
| Comparative | 97.5 | 95.4 | 89.8 | 0.06 | 8.83 | 15.30 | 0.03 | 5.98 | 10.50 | Clear | Clear | Clear |
| Example 1 | | | | | | | | | | Transparent | Transparent | Transparent |
| pH6 | | | | | | | | | | | | |
| Comparative | 95.5 | 94.3 | 90.1 | 0.06 | 8.96 | 15.70 | 0.04 | 6.09 | 10.70 | Clear | Clear | Clear |
| Example 1 | | | | | | | | | | Transparent | Transparent | Transparent |
| pH6.5 | | | | | | | | | | | | |
| Comparative | 96.4 | 93.4 | 89.9 | 0.07 | 9.26 | 15.80 | 0.04 | 6.29 | 10.80 | Clear | Clear | Clear |
| Example 1 | | | | | | | | | | Transparent | Transparent | Transparent |
| pH7 | | | | | | | | | | | | |
| Comparative | 94.8 | 91.4 | 86.4 | 0.10 | 10.80 | 18.90 | 0.07 | 7.25 | 12.60 | Clear | Faint | Faint |
| Example 1 | | | | | | | | | | Transparent | yellow | yellow |
| pH7.35 | | | | | | | | | | | Transparent | Transparent |

The experimental results showed that the formulations of Examples 1 to 5 had no significant changes in the content of the active ingredient, the content of related substances, and the color and clarity of the solutions within 2 months under the accelerated test conditions, indicating good stability. In contrast, the formulation of Comparative Example 1 showed a significant decrease in the content of the active ingredient (about 10%), a significant increase in the content of the maximum single impurity of related substances (about 10%), and a significant increase in the content of the total impurities of related substances within 2 months under the accelerated test conditions, indicating poor stability.

### Test on Example 1: Pharmacokinetics Study in Plasma and Ocular Tissues of Rabbits after Ocular Administration of Formulations of Comparative Example 1 (pH 6.5) and Example 1

Experimental method: 42 male rabbits were divided into 3 groups. Group 1 consisted of 16 rabbits, each dropped with 20 μL of the formulation of Example 1 in both eyes. Group 2 consisted of 16 rabbits, each dropped with 20 μL of the formulation of Comparative Example 1 in both eyes. Group 3 consisted of 10 rabbits, each injected intravenously via the ear margin with 5 mg/kg of the formulation of Comparative Example 1. Carbon dioxide euthanasia was performed after blood collection via the middle ear artery in the ear at 0.25, 1, 2, 3, 4, 6, 8, and 10 hours after administration for Group 1; at 0.25, 1, 1.5, 2, 2.5, 3, 4, and 5 hours after administration for Group 2; and at 0.5, 1, 2, 3, and 4 hours after administration for Group 3. The eyes were dissected and the corresponding ocular tissues were collected. The concentrations of voriconazole in whole blood and ocular tissue samples were determined by LC-MS/MS.

| | | Administration | Dose (mg/ | Animal |
|---|---|---|---|---|
| Group | Sample name | route | rabbit) | amount |
| 1 | Example 1 (Voriconazole | Drop to eyes | 20 μL/eye | 16 |
| | eye drop) | | | |
| 2 | Comparative Example 1 | Drop to eyes | 20 μL/eye | 16 |
| | (Voriconazole injection) | | | |
| 3 | Comparative Example 1 | Injection | 5 mg/kg | 10 |
| | (Voriconazole injection) | | | |

The experimental results showed that topical intraocular administration increased the concentration of voriconazole in the ocular tissues, and achieved the goal of active ingredient enrichment in the eyes, compared with intravenous administration. Meanwhile, the drug exposure in plasma was lower after topical intraocular administration, which can avoid adverse effects caused by systemic administration.

The plasma exposure was lower in aminals in both Group 1 and Group 2 after topical intraocular administration of 20 μL/eye of the formulation of Example 1 and the formulation of Comparative Example 1, respectively. The conjunctival, corneal, and target tissue (aqueous humor) exposures and half-life were higher in Group 1 than in Group 2. After intravenous injection of 5 mg/kg of the formulation of Comparative Example 1 via the vein of the ear margin, the animals in Group 3 showed a higher plasma exposure, a conjunctival exposure comparable to that in Group 2, and lower exposures in remaining ocular tissues than those in both Group 1 and Group 2.

The main pharmacokinetic parameters were shown in the table below:

| Sample | Parameters | Unit | Group 1 | Group 2 | Group 3 |
|---|---|---|---|---|---|
| Plasma | AUC₍₀₋ₜ₎ | ng/mL*h | 24.526 | 14.944 | 868.652 |
| | t_{1/2z} | h | 3.416 | 1.223 | 0.519 |
| | Tₘₐₓ | h | 0.250 | 0.250 | 0.500 |
| | Cₘₐₓ | ng/mL | 14.577 | 15.315 | 802.945 |
| Conjunctiva | AUC₍₀₋ₜ₎ | ng/g*h | 1243.457 | 700.630 | 690.870 |
| | t_{1/2z} | h | 2.820 | 1.102 | 0.643 |
| | Tₘₐₓ | h | 0.250 | 0.250 | 0.500 |
| | Cₘₐₓ | ng/mL | 720.473 | 703.143 | 685.890 |
| Cornea | AUC₍₀₋ₜ₎ | ng/g*h | 1006.171 | 780.980 | 462.448 |
| | t_{1/2z} | h | 3.417 | 1.205 | 0.768 |
| | Tₘₐₓ | h | 0.250 | 0.250 | 0.500 |
| | Cₘₐₓ | ng/mL | 944.190 | 892.978 | 410.789 |
| Aqueous | AUC₍₀₋ₜ₎ | ng/mL*h | 1408.899 | 858.211 | 627.177 |
| humor | t_{1/2z} | h | 2.422 | 0.954 | 0.647 |
| | Tₘₐₓ | h | 0.250 | 0.250 | 0.500 |
| | Cₘₐₓ | ng/mL | 2214.245 | 1324.140 | 610.447 |

1. A pharmaceutical composition, comprising: a triazole antifungal agent as an active ingredient, a non-aqueous solvent, and optionally a tonicity regulator, and preferably, the pharmaceutical composition is a non-aqueous preservative-free composition.

2. The pharmaceutical composition according to claim 1, wherein the triazole antifungal agent is selected from the group consisting of fluconazole, voriconazole, and isavuconazole.

3. The pharmaceutical composition according to claim 1, wherein the non-aqueous solvent is a glyceride of a C₆-C₁₂ fatty acid, or the non-aqueous solvent contains a glyceride of a C₆-C₁₂ fatty acid.

4. The pharmaceutical composition according to claim 1, wherein the non-aqueous solvent has an ester bond (—C(O)—O—) content of about 20.0% to about 35.0% by molecular weight, or about 22.0% to about 28.0% by molecular weight.

5. The pharmaceutical composition according to claim 3, wherein the non-aqueous solvent has an ester bond (—C(O)—O—) content of about 20.0% to about 35.0% by molecular weight, or about 22.0% to about 28.0% by molecular weight.

6. The pharmaceutical composition according to claim 3, wherein a fatty acid moiety of the glyceride of the C₆-C₁₂ fatty acid is one or more selected from the group consisting of hexanoic acid, heptanoic acid, octanoic acid, nonanoic acid, decanoic acid, undecanoic acid, and dodecanoic acid; or the glyceride of the C₆-C₁₂ fatty acid is one or more selected from the group consisting of medium chain triglycerides, glycerol trihexanoate, glycerol trioctanoate, glycerol tridecanoate, and glycerol tridodecanoate.

7. The pharmaceutical composition according to claim 1, further comprising the tonicity regulator, such as cyclomethicone.

8. The pharmaceutical composition according to claim 3, further comprising the tonicity regulator, such as cyclomethicone.

9. The pharmaceutical composition according to claim 4, further comprising the tonicity regulator, such as cyclomethicone.

10. The pharmaceutical composition according to claim 6, further comprising the tonicity regulator, such as cyclomethicone.

11. The pharmaceutical composition according to claim 7, wherein the cyclomethicone is a fully methylated cyclosiloxane containing a repeating unit represented by a molecular formula of [—(CH₃)₂SiO-]n, where n is 4, 5, or 6, or a mixture of the foregoing.

12. The pharmaceutical composition according to claim 8, wherein the cyclomethicone is a fully methylated cyclosiloxane containing a repeating unit represented by a molecular formula of [—(CH₃)₂SiO-]n, where n is 4, 5, or 6, or a mixture of the foregoing.

13. The pharmaceutical composition according to claim 9, wherein the cyclomethicone is a fully methylated cyclosiloxane containing a repeating unit represented by a molecular formula of [—(CH₃)₂SiO-]n, where n is 4, 5, or 6, or a mixture of the foregoing.

14. The pharmaceutical composition according to claim 10, wherein the cyclomethicone is a fully methylated cyclosiloxane containing a repeating unit represented by a molecular formula of [—(CH₃)₂SiO-]n, where n is 4, 5, or 6, or a mixture of the foregoing.

15. The pharmaceutical composition according to claim 1, wherein the pharmaceutical composition comprises about 0.2% (w/v) to about 2% (w/v) of the triazole antifungal agent, about 80% (v/v) to about 100% (v/v) of the non-aqueous solvent, and about 0% (v/v) to about 20% (v/v) of the tonicity regulator.

16. The pharmaceutical composition according to claim 3, wherein the pharmaceutical composition comprises about 0.2% (w/v) to about 2% (w/v) of the triazole antifungal agent, about 80% (v/v) to about 100% (v/v) of the non-aqueous solvent, and about 0% (v/v) to about 20% (v/v) of the tonicity regulator.

17. The pharmaceutical composition according to claim 4, wherein the pharmaceutical composition comprises about 0.2% (w/v) to about 2% (w/v) of the triazole antifungal agent, about 80% (v/v) to about 100% (v/v) of the non-aqueous solvent, and about 0% (v/v) to about 20% (v/v) of the tonicity regulator.

18. The pharmaceutical composition according to claim 6, wherein the pharmaceutical composition comprises about 0.2% (w/v) to about 2% (w/v) of the triazole antifungal agent, about 80% (v/v) to about 100% (v/v) of the non-aqueous solvent, and about 0% (v/v) to about 20% (v/v) of the tonicity regulator.

19. The pharmaceutical composition according to claim 7, wherein the pharmaceutical composition comprises about 0.2% (w/v) to about 2% (w/v) of the triazole antifungal agent, about 80% (v/v) to about 100% (v/v) of the non-aqueous solvent, and about 0% (v/v) to about 20% (v/v) of the tonicity regulator.

20. A method for treating eye diseases and ear diseases caused by fungal infections, comprising administering the pharmaceutical composition according to claim 1, wherein the eye diseases and ear diseases include fungal keratitis, otitis externa or otitis media caused by fungal infections.
